(19) United States
(12) Patent Application Publication
(10) Pub. No.: US 2008/0115793 A1
(43) Pub. Date: May 22, 2008

(54) METHODS AND DEVICES FOR ACCESSING THE HEART

(76) Inventor: Edmund J. ROSCHAK, Mission Viejo, CA (US)

Correspondence Address: LEVINE BAGADE HAN LLP, 2483 EAST BAYSHORE ROAD, SUITE 100, PALO ALTO, CA, 94303, US

(21) Appl. No.: 11/943,881

(22) Filed: Nov. 21, 2007

Related U.S. Application Data
(60) Provisional application No. 60/860,299, filed on Nov. 21, 2006.

(51) Int. Cl. A61B 17/00

(52) U.S. Cl. 128/898; 600/101

(57) ABSTRACT

Methods and devices are disclosed herein for treating organs within the thoracic cavity, such as the heart, by navigation through a natural respiratory opening and through a wall of an associated body lumen.

### CROSS-REFERENCE TO RELATED APPLICATIONS

The present application is a non-provisional of provisional application no 60/860,299 filed on Nov. 21, 2006. The entirety of which is incorporated by reference herein.

### BACKGROUND OF THE INVENTION

The present invention relates to accessing areas within the thoracic cavity, such as for surgical, diagnostic, or exploratory procedures, and to the particular field of accessing the heart during surgical procedures on or within the heart.

Several types of surgical procedures are currently performed to investigate, diagnose, and treat diseases of the heart and the great vessels of the thorax. Such procedures include repair and replacement of mitral, aortic, and other heart valves, repair of atrial and ventricular septal defects, pulmonary thrombectomy, treatment of aneurysms, electrophysiological mapping and ablation of the myocardium, coronary bypass grafts, and other procedures in which interventional devices are introduced into the interior of the heart or a great vessel.

By way of background, the basic operation of a heal will be briefly discussed. The heart works like a pump. The left and right ventricles are separate but share a common wall (the septum). The left ventricle is thicker and pumps the blood into the systemic circulation. The work it performs is much greater than the right ventricle. The right ventricle pumps blood into the pulmonary circulation, which is a low pressure circuit. The left ventricle wall (a low energy system) is much thinner than the right ventricle.

The left ventricle fills in diastole and ejects in systole. The difference between the diastolic volume (largest) and the systolic volume (smallest) (the stroke volume or amount of blood ejected on each heartbeat) multiplied by heart rate determines the cardiac output of the heart (liters/min. of flow). The heart shortens during systole as the muscle contracts. There are a number of motions during contraction (including a considerable amount of rotation) but for practical purposes the heart can be thought of as a truncated cone. The heart functions well whether the person is upright, upside down, prone or supine. It sits inside the pericardium—a sac which limits its motion and spreads the support on the heart so that no matter how a person positions himself, it is not particularly compressed and is able to fill and then eject with each heartbeat.

Using traditional techniques, many procedures require a gross thoracotomy, usually in the form of a median sternotomy, to gain access into the patient's thoracic cavity. A saw or other cutting instrument is used to cut the sternum longitudinally, allowing two opposing halves of the anterior or ventral portion of the rib cage to be spread apart. A large opening into the thoracic cavity is thus created, through which the surgical team may directly visualize and operate upon the heart and other thoracic contents.

Surgical intervention within the heart often requires isolation of the heart and coronary blood vessels from the remainder of the arterial system, and arrest of cardiac function. Usually, the heart is isolated from the arterial system by introducing an external aortic cross-clamp through a sternotomy and applying it to the aorta between the brachiocephalic artery and the coronary ostia. Cardioplegic fluid is then injected into the coronary arteries, either directly into the coronary ostia or through a puncture in the aortic root, so as to arrest cardiac function. In some cases, cardioplegic fluid is injected into the coronary sinus for retrograde perfusion of the myocardium. The patient is placed on cardiopulmonary bypass to maintain peripheral circulation of oxygenated blood.

Using the open-chest technique described above, the large opening provided by a median sternotomy or right thoracotomy enables the surgeon to see the heart directly and to position his or her hands within the thoracic cavity in close proximity to the exterior of the heart for manipulation of surgical instruments and removal of excised tissue. However, these types of invasive, open-chest procedures produce a high degree of trauma, a significant risk of complications, an extended hospital stay, and a painful recovery period for the patient. Moreover, while open-chest heart surgery produces beneficial results for many patients, numerous others who might benefit from such surgery are unable or unwilling to undergo the trauma and risks of current techniques.

Recent advances in minimally invasive techniques enable accessing the thoracic cavity without the use of a thoracotomy. Following one approach, access is provided by several small incisions through the chest wall. A variety of devices, including a camera to allow visualization, are then inserted through these small incisions. These instruments are then manipulated to perform the intended treatment. After completing the procedure, the devices are removed and the small incisions are closed. Replacing the gross thoracotomy with several small incisions are thought to result in benefits to the patients through shorter stays in the hospital, faster recovery, less trauma mid blood loss, lower infection rates and less cost.

There are as many as 300,000 coronary bypass graft procedures performed annually in the United States. Each of those procedures may include one or more graft vessels. Until recently, coronary artery bypass procedures have been performed with the patient on cardiopulmonary bypass whereby the heart is stopped with cardioplegia and the surgery performed on an exposed and still heart.

Some pioneering surgeons are performing procedures in which the coronary bypass is performed on a beating heart. That is, without heart-lung bypass and cardioplegia. This minimizes the time it takes to perform the procedure and reduces the cost of the operation by eliminating the heart-lung bypass machine.

Coronary Artery Bypass Grafting (CABG) is performed and a new blood supply to the heart muscle is established when coronary arteries are blocked with calcium or plaque. A new blood supply conduit is joined to the diseased coronary, distal to the blockage, thus providing a fresh supply of oxygenated blood to the vessel in question. Today, this is accomplished by hand suturing a graft vessel (the new supply of blood) to the diseased vessel. This junction is called an anastomosis of vessels. Many different types of supply conduits can be used. Examples are cadaver vein, saphenous vein, radial artery, internal mammary artery, and the like.

When the chest is opened by a median sternotomy it is possible to gain access to all chambers and surfaces of the heart. This combined with the fact that this incision is usually less painful than a thoracotomy (rib separation), makes this the popular surgical approach to the heart.

The coronary vessels are surface vessels, only occasionally dipping into the myocardium making them accessible without opening the heart. Traditionally, bypass surgery is done with the heart arrested. This stops the motion of the heart and allows the arrest of the coronary circulation so the surgeon sews in a bloodless and easy to see field. Since the heart is stopped, the patient would suffer irreversible damage to the brain and other tissues and organs without the use of the heart-lung machine to support the general circulation. Although the heart-lung machine has been refined, it is particularly toxic to older and debilitated patients and it is expensive.

It is possible to perform surgery off bypass, while the heart is beating and the coronaries are under positive blood pressure; however, there may be problems. One problem is that not all vessels are accessible since some vessels are on the posterior or inferior surfaces and that when such vessels are brought into view by lifting the heart; cardiac performance is impaired such that the cardiac output falls and blood pressure drops. A second problem is that the heart moves so that suturing in vessels (12 to 15 stitches in a vessel under 2 mm in diameter) might be inaccurate and a third problem is that there is blood in the field as the coronary circulation is not interrupted. This last problem is now largely solved by snares, which temporarily stop the flow of blood through the targeted arteries. The problem of lifting the heart is not to impair the performance of the heart while at the same time adequately exposing the heart and regionally immobilizing the vessel during beating heart surgery, and this problem is not solved with any prior art system.

What is needed, therefore, are devices and methods for carrying out procedures on the heart and great vessels that reduce the trauma, risks, recovery time and pain that accompany current techniques. The devices and methods should facilitate surgical intervention within the heart or great vessels without the need for a gross thoracotomy, or even small incisions within intercostal spaces of the rib cage. In particular, the devices and methods should allow for removal of tissue from the thoracic cavity, as well as for introduction of surgical instruments and visualization devices. In addition, the devices and methods should provide access to heart surfaces without the need to significantly change the position of the heart within the thoracic cavity.

### SUMMARY OF THE MENTION

The description, objects and advantages of the present invention will become apparent from the detailed description to follow, together with the accompanying drawings.

The disclosure and invention specifically include combination of features of various embodiments as well as combinations of the various embodiments where possible.

This invention relates to devices and methods for accessing regions within the thoracic cavity by navigating one or more devices through a natural respiratory opening. In one variation, the procedure allows for access of the heart in order to execute one or more diagnostic or treatment procedures on the heart by inserting one or more devices and/or instruments through the mouth or nose. Alternatively, the procedure may be performed by passing a device through an opening in the trachea.

In particular, a variation of the methods described herein include the act of inserting a device or devices into the mouth or nose into a body lumen, positioning through the body lumen to a location near the heart but free of obstacles between the lumen and heart surface, creating an opening in the body lumen wall to access the mediastinum, navigating one or more devices from the opening to the surface of the pericardium, creating an opening in the pericardium, navigating one or more devices to the surface of the heart, deploying a device or devices to perform a heart diagnostic procedure and/or treat the surface of the heart and finally closing all openings when finished. If needing to perform a diagnostic or treatment procedure(s) within the heart, an opening in the surface of the heart will be created, a devices or device(s) will navigated into the intended portion of the heart, a procedure or procedures will be performed, the devices will then be removed and the opening will be closed when finished.

Two body lumens, the esophagus and/or trachea and bronchial tree, are accessible through the nose and mouth. These lumens may be used to navigate very near to the heart. This intracardiac access can be used to perform a variety of diagnostic and treatment procedures within the heart or great vessels without the need for any incisions in the chest wall including those used for gross thoracotomy, or keyholes or ports. In this way, an opportunity exists to conduct procedures within or in direct contact with the heart and avoid the pain and recovery associated with techniques that access the heart through the chest wall.

A variation of the invention includes the use of an endoscope capable of traversing through the mouth, either the esophagus or the trachea and bronchial tree, and beyond through the breach in either lumen wall, from the lumen wall to the pericaridum and finally through an access point in the pericardium to access the heart.

Depending on the intended diagnostic or surgical procedure, interventionalists will determine the optimal access point through the pericardium to reach the intended region or surface of the heart. The pericardium is a double membrane structure containing a serous fluid to reduce friction during heart contractions. The mediastinum, a subdivision of the thoracic cavity, is the name of the heart cavity. Using this point on the pericardium as a target, the esophagus or tracheobronchial tree will be selected based oil the path of least resistance. This determination will include a consideration of orientation to and distance from the pericardium target. For example, for anterior pericardial targets the tracheobronchial tree may provide the best access. Similarly, for posterior pericardial targets, the esophagus may be more appropriate.

After selecting the lumen, the approximate location of exit from the lumen will be estimated. Another part of the selection of the exit location will involve an understanding of the tissues or structures between the body lumen and the heart. The primary obstacles to be avoided are blood vessels. Both systemic and pulmonary blood vessels may be found between the natural body lumens and the heart. For example, within the pericardium are the proximal ends of the aorta, vena cava, and the pulmonary artery. The mediastinum contains many blood vessels as well. However, in most cases, paths can be identified that avoid interacting with these obstacles. The primary technique to overcome these obstacles will be avoidance since paths can be found that above or below major collections of blood vessels. For example, an exit point from tracheobronchial tree may be best at the main carina moving inferiorly due to lack of blood vessels in this path to the heart. As a result, in most cases special catheters that help avoid blood vessels will not be required.

To improve the likelihood of selecting vessel free paths, three-dimensional radiologic modeling may be used to provide a path from the mouth, through the natural lumen and finally to the target. This path or map may be used during the procedure to help guide the interventionalist using a registration system to link the events within the patient to the three-dimensional map.

Following this pre-procedure planning, the patient is anesthetized and intubated. Using a steerable endoscope-like instrument through the mouth or nose, the interventionalist accesses the selected natural lumen and drives the endoscope to the intended location of the lumen breach.

An important feature of this invention is the method of creating, first the breach in the lumen wall, and then the breach in the pericardium to access the heart surface. Finally, a breach in the heart is required if the goal is to conduct procedures within the heart. These breaches may be completed by a single device or separate devices. In either case, these breaches need to be made with great care to avoid inadvertently damaging unintended tissues or obstacles but be large enough to allow passage of the endoscope. The breach may be made by any of the well known mechanical means like dilation, cutting, piercing, or bursting. The use of electrical energy, commonly delivered during surgical procedures by a radio frequency generator, will also result in acceptable lumen breaches as well. In addition, breaches may be created using alternative energy sources. For example, chemicals, ultrasound, laser, microwave, and cryoablation energy may be used to create a lumen breach. It is anticipated that the initial breach may be small and therefore require expansion to create clearance to allow the endoscope through the breach. This expansion may be created though the use of a balloon device.

The invention further includes the use of a device or devices to identify the location of these blood vessels prior to, during, or after the creation of the breach. This avoidance may be accomplished by the use of non-invasive imaging such as radiography, computed tomography (CT) imaging, ultrasound imaging, Doppler imaging, acoustical detection of blood vessels, pulse oximetry or thermal detection or imaging. The avoidance may also be accomplished using Doppler effect, for example transmission of a signal which travels through tissue and other bodily fluids and is reflected by changes in density that exist between different body tissues or fluids. If the signal is reflected from a tissue/fluid that is moving relative to the sensor, then the reflected signal is phase shifted from the original signal thereby allowing for detection.

Another variation of the invention includes the act of sealing any or all of the breaches. In healthy condition, the body lumens and the heart are sealed from the intermediate region, the mediastinum. Although the devices used to pass through the breaches may provide any necessary sealing, it is anticipated that additional sealing may be required to keep fluids and pressures at appropriate levels. This sealing may be accomplished through the use of sleeves that tightly grip the opening and devices. These sleeves may be made of materials known in the art such as metals, polymers or biodegradables. One variation of these sleeves is the use of materials that swell to provide the seal when exposed to saline or common body fluids. Another variation is the use of a balloon or inflatable sleeve to provide a seal between the openings and the device(s). A balloon may be mounted on a dedicated device to provide sealing or the balloon can be mounted directly on the endoscope. It is anticipated that a single balloon may be capable of sealing all the openings or a series of dependant or independent balloons may be used. The above mentioned sleeves or balloons may also provide anchoring to stabilize the devices during use.

A further variation of the invention includes the act of moving, dissecting or removing tissue or fluid in the lumen, mediastinum aid heart to allow passage of the device or devices through the openings and into position at the heart surface or within the heart. Tissue may be moved through blunt or balloon dissection. If necessary, after moving or dissecting the tissue, a structure, either mechanically or balloon deployed, may be positioned to maintain the dissection or working space to manipulate the devices and visualize the remaining steps. For example, a structural balloon may enter the medialstinal space as part of a small diameter catheter but then can be deployed using saline to produce rigid structure that creates a 10-20 cm3 working space. In similar fashion, a constrained shape memory material may be inserted into the dissected space and then released to self-expand to contain a similar volume due to the exposure to body temperature.

When the procedure is finished, cold saline may be introduced to soften the material to enable removal. Cutting and grasping devices may be used to remove materials. In addition, ablation techniques such as radio frequency or cryo are used commonly to remove material. The catheters appropriate for this tissue dissection or removal need to fit through the working channel of the endoscope, have sufficient flexibility to allow passage through tortuous non-linear paths and maintain adequate column strength apply force against the tissue of interest. In addition, these catheters may benefit from the ability to steer them independent of the endoscope. In this way, the endoscope may be used to provide access and sealing to the initial opening in the lumen. The catheters would then be used to access the mediastinum, pericardium and heart. The ability to steer the catheters makes successful catheter deployment much more likely. Alternatively, the endoscope may be used all the way to the heart and steering the catheters may not be required.

Another variation of the invention includes the act of closing the openings following completion of the diagnostic and/or treatment procedure(s). A variety of techniques may be employed to achieve closure such as sutures, absorbables, staples, clips, tapes and adhesive compounds. Octyl-2-cyanoacrylate (Dermabond, Ethicon, Somerville, N.J.) is a cyanoacrylate tissue adhesive approved by the U.S. Food and Drug Administration (FDA) for superficial skin closure. Fibrin-based tissue adhesives can be created from autologous sources or pooled blood. They are typically used for hemostasis and can seal tissues. Fibrin tissue adhesives can be used to fixate skin grafts or seal fluid leaks. Commercial preparations such as Tisseel (Baxter) and Hemaseel (Haemacure) are FDA-approved fibrin tissue adhesives made from pooled blood sources. These fibrin tissue adhesives are relatively strong and can be used to fixate tissues. Autologous forms of fibrin tissue adhesives can be made from patient's plasma. Tissue sealants and gels are also well known and commonly used to aid wound healing of surgical incisions. Collagen and platelet gels are known to improve wound closure and strength. Similar materials viscous enough to fill the remaining opening and create a plug will be used. This plug may contain fibrin, growth factors (platlet derived, EGF, TGF-Beta, etc.), hypoxia inducible factors, connective tissue growth factors, etc. to promote wound healing.

Tissue grafts or patches may also be employed as well. Fibrin tissue patches have been shown to seal openings in the esophagus and thoracic cavity. Expanded polytetrafluoroethylene (ePTFE) is also used with success for soft tissue repairs like reconstruction of hernias. Other polymer and heterologous materials may also aid the closure of openings. For example, porcine pericardium has been used with success in treating pericardial defects.

Drugs have also been shown to aid wound healing throughout the body and are expected to be effective in this application as well. Those showing an activity of promoting wound healing, include the extract of aloe, antibiotics, anti-inflammatory agents, kallikrein, adenine, nicotinic acid, allantoin, vitamin A, zinc, c-AMP derivatives (Japanese Patent Application KOKAI No. 107935/1988), exogenous DNA (Japanese Patent Application KOKAI No. 505888/1988) and aganocides. In addition, growth factors such as TGF-beta, epithelial growth factors, platelet derived growth factors have also been shown to aid wound healing. All of the above techniques to promote closure of the openings may be used independently or in combination with one or more of the techniques described above.

Given the above techniques to aid closure of the openings and the strong healing response observed in the lungs and gastrointestinal track, healing the openings is not expected to be a significant challenge. Experience in the interventional pulmonary field supports this expectation. Breaches through the airway wall are made routinely to sample lymph nodes and tumors in the parenchyma. These breaches are known to heal within days and become unrecognizable as a wounded surface. In the esophagus, tissue glues and stents are commonly used to treat tracheo-esophageal fistulas with good success. Similarly, stents may be used to aid closure of the openings in the esophagus or tracheobronchial tree. In addition to the traditional metallic, polymer or polymer coated metallic stents, biodegradable stents may be particularly appropriate for this short-term wound healing application.

One area of concern with this inventive technique is the potential for inducing a pneumomediastinum. It occurs when air leaks from any part of the lung or airways into the mediastinum. Pneumomediastinum may not be accompanied by any symptoms. Usually, it causes severe chest pain below the sternum (breastbone) that may radiate to the neck or arms. The pain may be worse with breathing or swallowing. Often, no treatment is required as the air is gradually absorbed from the mediastinum. If pneumomediastinum is accompanied by pneumothorax, a chest tube may be placed. Chest tubes are commonly placed with excellent success in most incidences of pneumothorax. Breathing high concentrations of oxygen may allow the air in the mediastinum to be absorbed more quickly. To mitigate this risk, the treatment steps may include techniques to remove any air present in the mediastinum prior to closure of the opening in the trachea or esophagus.

Once access is achieved through) the mouth, another variation of the invention is the use of devices in combination with the endoscope to perform the diagnostic or treatment procedures on the heart. Procedures anticipated are repair and replacement of mitral, aortic, and other heart valves, repair of atrial and ventricular septal defects, pulmonary thrombectomy, treatment of aneurysms, endocardial ablation, electrophysiological mapping and ablation of the myocardium, coronary bypass grafts, and other procedures.

One specific procedure that might benefit from the present invention is endocardial ablation. Endocardial ablation is commonly used to treat disorders of the heart such as general arrhythmias, ventricular tachycardia, atrial fibrillation, atrial flutter, and Wolff-Parkinson-White Syndrome (WPW). Typically, ventricular tachycardia and WPW are treated by RF coagulation or DC discharge applied to cardiac tissue by electrode-tipped, deformable, and preset curved catheters. These catheters are of similar construction to those used in the art for electrically mapping the heart. In order to navigate through the patient's vascular system, cardiac catheters are limited to small diameters. A typical mapping or ablation catheter has small electrodes mounted on the distal end of the catheter shaft. In an alternative approach a balloon catheter is inflated with fluid within the coronary sinus and is heated by a heating device located within the balloon. Tissue surrounding the balloon is ablated by thermal conduction from the fluid to the tissue through the wall of the balloon. Both of these catheter types may be used with the present invention where access to the heart or within the heart is provided through a respiratory opening rather than percutaneously.

Another aspect of the invention involves surgical robotic systems. Robotics are being used in surgical procedures because they provide unprecedented control and precision of surgical instruments in minimally invasive procedures. These systems give the surgeon the ability to perform more complex surgical procedures than can be accomplished by traditional endoscopic surgery. In a similar way, robotics may be used in combination with the present invention to provide additional control throughout some or all steps in this method. Controlling the distal portions of the catheters or endoscopes to execute the therapeutic or diagnostic procedures may prove to be a challenge and incorporating robotic control systems may enable these methods to be successfully accomplished by a broader range of surgeons. Methods are anticipated in which the control and precision of robotic instruments described by patents and publications similar to U.S. Pat. Nos. 7,025,064 and 7,090,683, which are incorporated here by reference, are applied to the present inventive method.

### BRIEF DESCRIPTION OF THE DRAWINGS

FIG. 1 is an anterior view of the heart, lungs and trachea.

FIG. 2 is a posterior view of the heart, lungs and trachea.

FIG. 3 is a lateral view of the thoracic cavity showing die position of the esophagus and tracheobronchial tree relative to the heart.

FIG. 4 is a cross-sectional illustration of a human thorax.

FIG. 5 is an illustration showing two flexible endoscopes inserted through the mouth providing access to manipulate devices.

FIG. 6 is an illustration of a flexible endoscope.

FIG. 7 is an illustration of another flexible endoscope.

FIG. 8 is a transverse cross-sectional view of a patient's chest showing an alternative technique of accessing the heart.

FIG. 9 illustrates a method of accessing an internal chamber of the heart.

FIG. 10A illustrates an alternative method of accessing an internal chamber of the heart.

FIG. 10B illustrates an additional variation accessing an internal chamber of the heart using multiple openings and devices.

FIG. 11 illustrates a method of breaching an esophageal or tracheobronchial wall to access the mediastinum.

FIG. 12 illustrates the resulting breach in an esophageal or tracheobronchial wall.

FIG. 13 shows a balloon catheter deployed from an endoscope in the trachea creating a dissecting tissue in the mediastinum to create a working space.

### DETAILED DESCRIPTION OF THE INVENTION

Central to this invention, in all of its aspects, is the production ad maintenance of access pathways to regions within the thoracic cavity by using a natural respiratory opening. The esophagus and trachea and upper airways are positioned in the mediastinum in close proximity to the pericardial sac and heart. This inventive method takes advantage of this positioning to enable a minimally invasive, low trauma method of accessing the heart for diagnostic and therapeutic benefit.

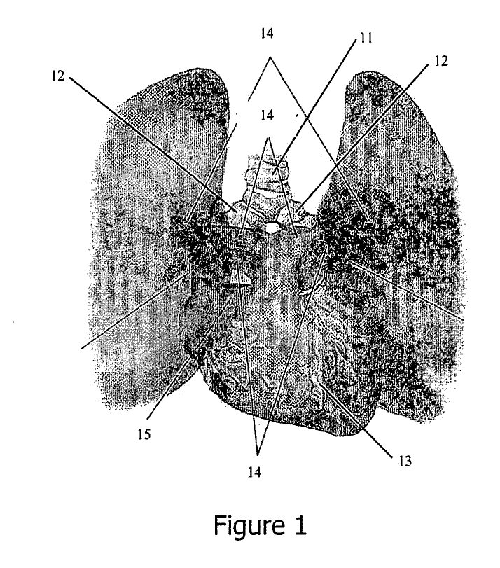

The location of the esophagus, trachea and upper airways relative to the heart is shown in FIGS. 1 through 4. FIG. 1 illustrates that the trachea 111 and upper airways 12 are posterior and superior to the heart 13. Utilizing the trachea and upper airways the anterior and superior surfaces of the heart may be accessed. It is clear that any path to the heart from the trachea and upper airways must incorporate steps to avoid the great vessels of the heart—the pulmonary vessels 14, aorta 15 and vena cava (not shown). Although locations exist where the breach in the trachea or upper airways can be created away from these vessels, most paths to the superior or anterior surfaces of the heart will require passage around these vessels. As long as this is accomplished without the use of sharp-tipped catheters, this should not be a problem.

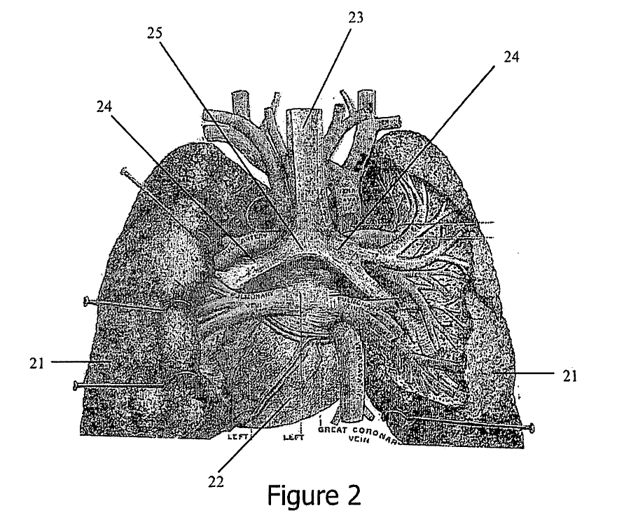

In this posterior view of the lungs 21 and heart 22, FIG. 2 provides a clear indication that the trachea 23 and upper airways 24 can also be used to access the posterior surface of the heart. At the same time, the esophagus, which is not shown in this view, can be used to conveniently access the posterior surface as well. For clarity, the esophagus is just proximal to the tracheo and upper airways and thus just out of the plane of this image. This illustration further shows that adequate space exists at the inferior surface of the main carina 25 of the tracheobronchial tree to create a safe opening and access the mediastinum.

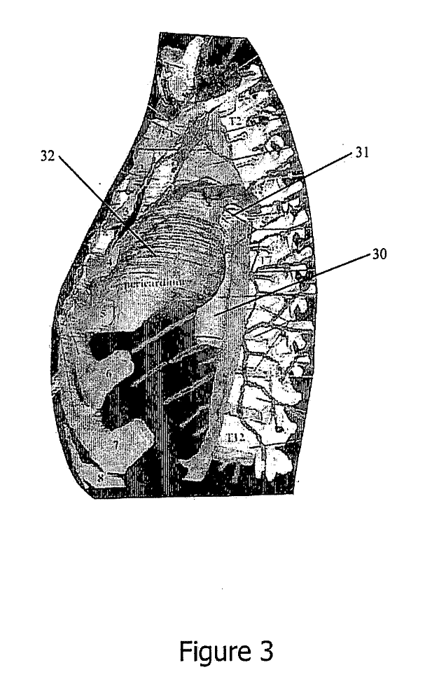

FIG. 3 is a lateral view of the thoracic cavity and provides a view of the esophagus 30, the tracheobronchial tree 31 and the pericardial sac 32. This view clearly demonstrates the posterior position of the esophagus relative to the tracheobronchial tree and pericardial sac.

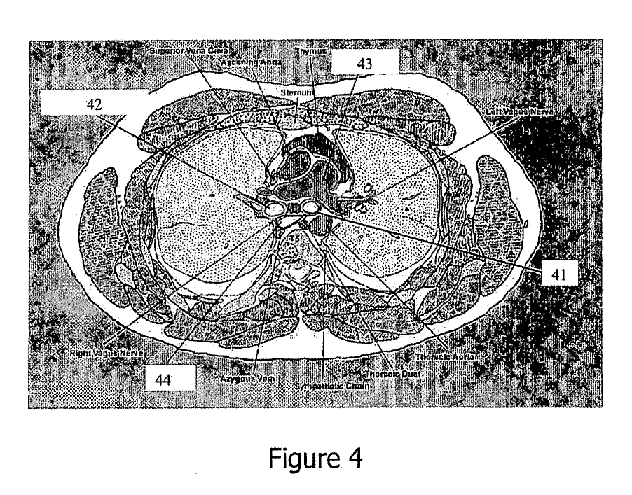

FIG. 4, a cross-sectional illustration of a human thorax, further illustrates the superior position of the main carina (not shown) of the tracheobronchial tree. In this view the left 41 and right main 42 bronchi are shown in plane with the pulmonary trunk 43. It is now clear that the main carina (not shown) is superior to, or above, the plane of this image. The esophagus 44 is also clearly posterior to the left and right main bronchi.

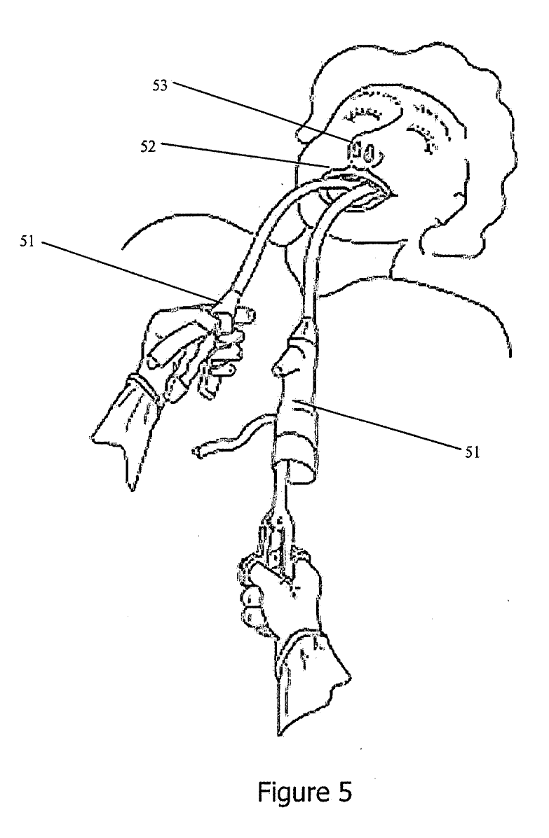

FIG. 5 demonstrates the manipulation of instruments 51, devices or endoscopes, through the mouth 52. It is clear that manipulation is also possible when using the nose 53 to access the esophagus (not shown) or tracheobronchial tree (not shown). Visualization is provided from optics located near the distal tip (not shown) of any of these instruments or devices and is projected onto a monitor (not shown) within the procedure room.

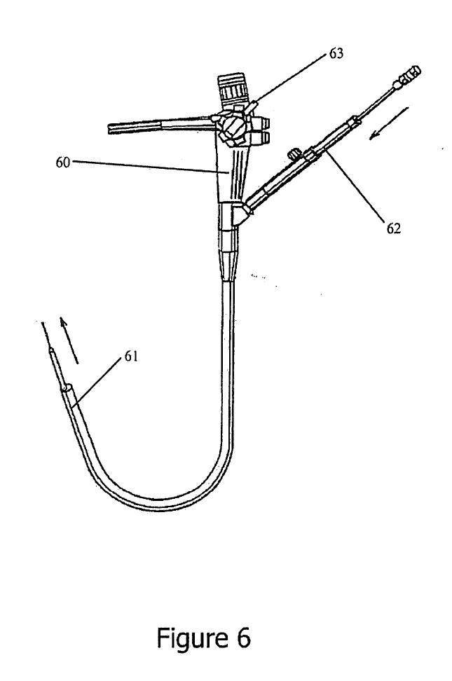

FIG. 6 is an illustration of an example endoscope 60 used in FIG. 5. This device is steer-able, has optics on the distal tip 61 for visualization and a working channel to allow passage of catheter devices 62 meant for use in a diagnostic or therapeutic procedure. Articulation is accomplished through the use of the thumb lever 63 located near the proximal end of the endoscope.

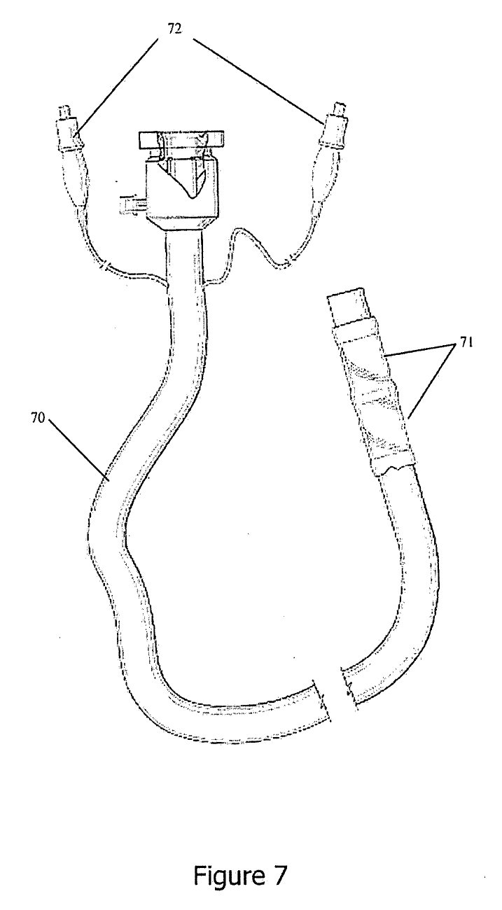

FIG. 7 is a drawing of an alternative endoscope 70 showing an inflatable feature near the distal tip 71 that may be used to seal the scope against an opening or anchor the scope in place. This has two independent inflatable chambers. Each of these chambers may be inflated using the two connections or valves 72 located at the proximal end of the scope.

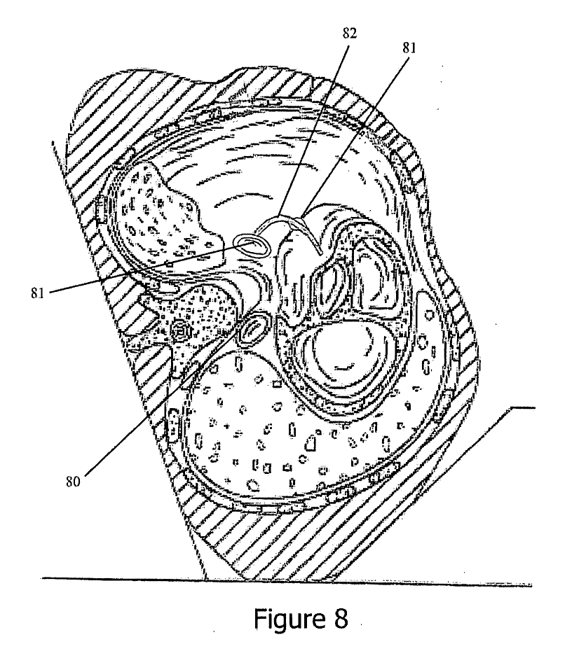

FIG. 8 provides an axial view of the thoracic cavity. This view shows both the right main bronchus 80 and the left main bronchus 81. A catheter 82 is shown exiting the left main bronchus and following a path to the surface of the heart. The inflatable portion 83 maintains a seal between the mediastinum and the pleural cavity. In this alternative approach, the left lung has been deflated to create a working space for the endoscope and instruments. This may or may not be required depending on the intended procedure and desired heart access point.

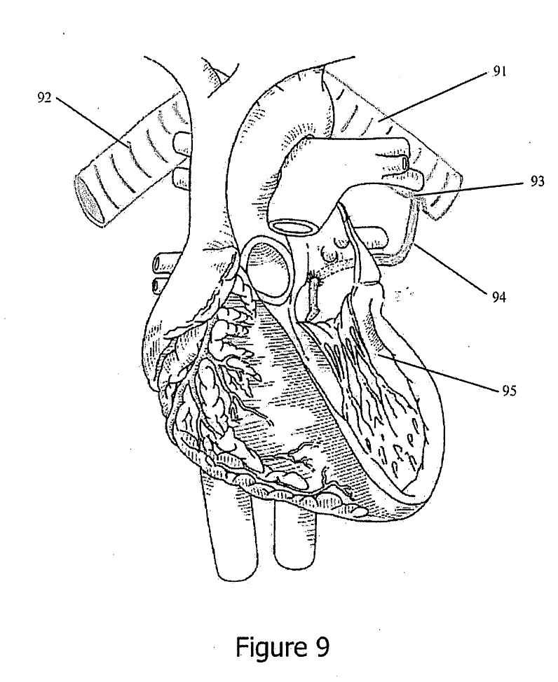

FIG. 9 shows an anterior cut-away view of the heart. The left 91 and right 92 main bronchi are shown posterior to the heart. Exiting from an opening 93 in the left main bronchus 91 is a catheter or endoscope 94. This catheter or endoscope is then shown entering the left side of the heart 95 to perform a procedure within the heart.

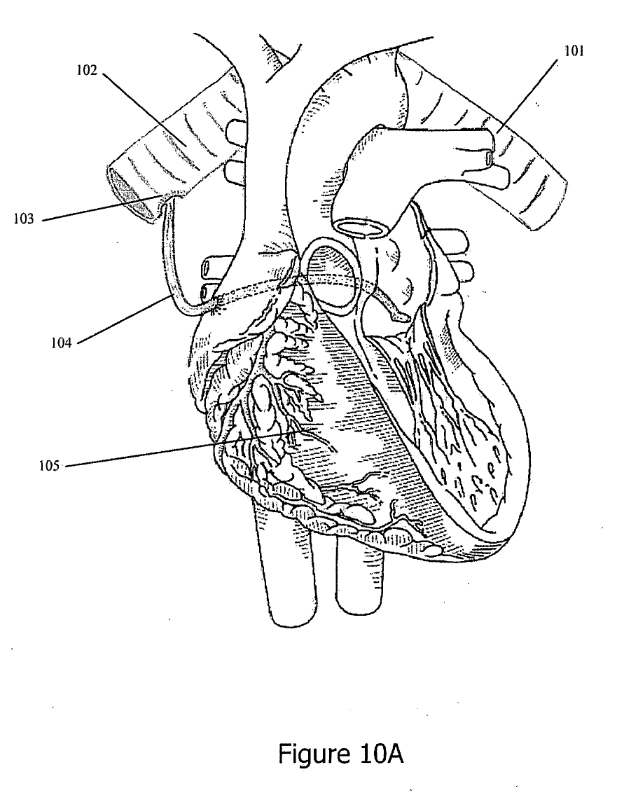

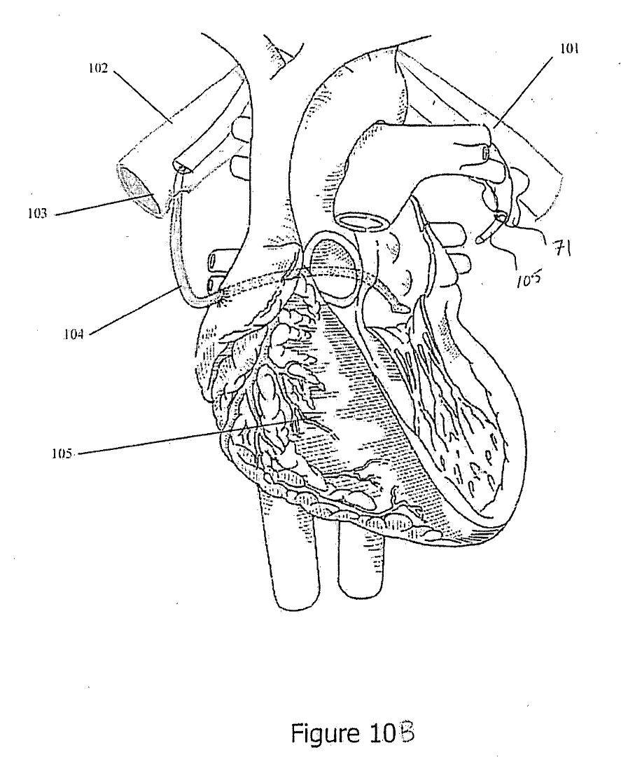

FIG. 10A is another anterior cut-away view of the heart. The left 101 and right 102 main bronchi are shown posterior to the heart. Exiting from an opening 103 in the right main bronchus 102 is a catheter or endoscope 104. This catheter or endoscope is then shown entering the right side of the heart 105 to perform a procedure within the heart. In this case the device enters the right side of the heart but is advanced into the left side. In other instances the device may remain in the right side to perform the therapeutic or diagnostic procedure.

FIG. 10B shows a cross sectional view of the left 101 and right 102 main bronchi to illustrate a second device 104 being accessed through the bronchi to an area adjacent to the heart 105. Although not required, a second device can be accessed within the left 101 or right 102 main bronchi. Alternatively, one or more devices can be advanced through a single bronchi or through the esophagus to perform the desire procedures. As noted above, an opening in the airway (or esophagus) can be sealed with a balloon-type member. Alternatively, the opening can be dilated to allow for passing of various devices therethrough.

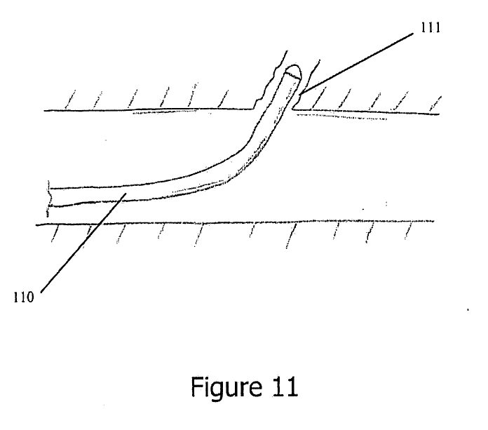

FIG. 11 shows a catheter 110 used to breach the esophagus or tracheobronchial lumen 111. As discussed previously, the step of creating the opening may be accomplished through a method selected from known mechanical, electrical, microwave, laser, thermal or chemical techniques.

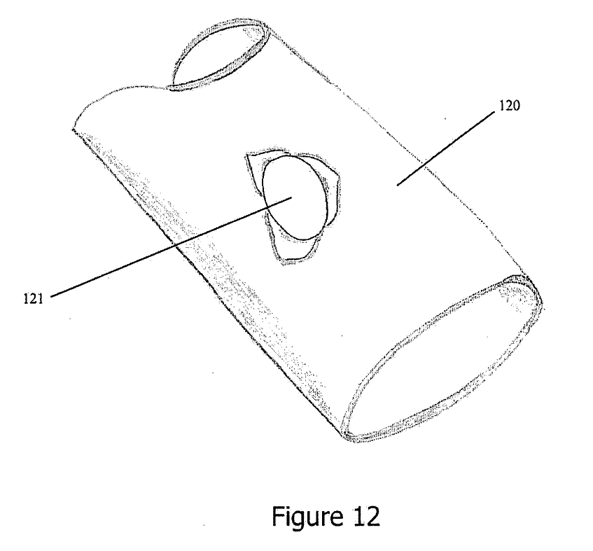

FIG. 12 shows one possible resulting breach 121 in the lumen 120. As discussed previously, the step of closing the opening may be accomplished using a variety of techniques such as patches, growth factors and drugs to aid wound closure following the procedure.

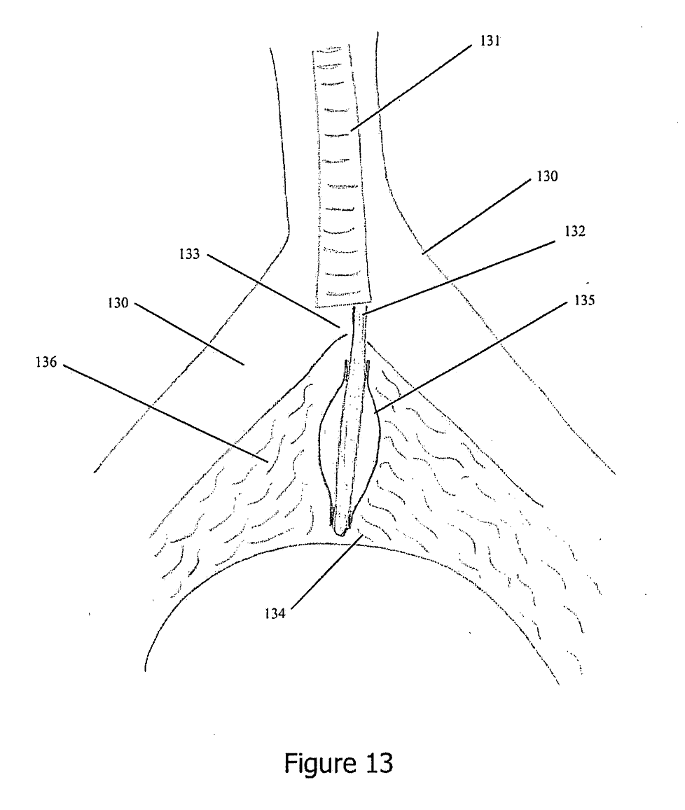

FIG. 13 shows an endoscope 131 advanced in the tracheobronchial tree 130. Deployed from the endoscope is a catheter 132 exiting through an opening 133 in the main carina and advancing toward the pericardium 134. An inflatable portion 135 is shown creating a working space in the tissues within 136 the mediastinum. In this embodiment, once the working space is created the endoscope may be advanced into the mediastinum to allow visualization of the pericardium before the next opening is created.

The invention herein is described by examples and a desired way of practicing the invention is described. However, the invention as claimed herein is not limited to the specific description in any manner. Equivalence to the description as hereinafter claimed is considered to be within the scope of protection of this patent.

I claim:

1. A method of accessing a region of the heart, the method comprising:
inserting a device through a natural respiratory opening a body lumen;
positioning the device through the body lumen to a location near the heart but within the body lumen;
creating an opening in a wall of the body lumen;
navigating one or more devices from the opening to the surface of the heart performing a procedure at the region of the heart with the therapeutic device; and
withdrawing the medical device from the natural respiratory opening and closing the opening in the body lumen.

2. The method of claim 1 where the lumen is an esophagus.

3. The method of claim 1 where the lumen is an airway.

4. The method of claim 1 where positioning through the body lumen to the location near the heart but within the body lumen comprises positioning a working end of an endoscope to the location.

5. The method of claim 1 where the creating step further involves sealing the opening to keep fluid from flowing into or out of the opening.

6. The method of claim 1 where the second navigation step involves the creation of artificial pathways from the lumen wall to the surface using tissue dissection or removal.

7. The method of claim 1 where the second navigation step involves the creation of a working space to allow deployment and manipulation of devices and visualize.

8. The method of claim 1 where the closure step is accomplished through a method selected from known mechanical, electrical, microwave, laser, thermal or chemical techniques.

9. The method of claim 1 where the closure step involves the application of one or more drugs to aid healing.

10. The method of claim 1 where the devices are manipulated-remotely or robotically.

11. The method of claim 1 where the surface of the heart includes the great vessels.

12. The method of claim 1 where more than one opening in a wall of a body lumen is created to execute the procedure.

13. The method of claim 1 where one or more openings are created in more than one body lumen to execute the procedure.

14. The method of claim 1 further comprising creating an opening in the surface of the heart prior to performing the procedure at the region of the heart.

15. The method of claim 14 where performing the procedure at the region of the heart comprises performing the procedure within the heart.

16. The method of claim 14 where the lumen is the esophagus.

17. The method of claim 14 where the lumen is an airway.

18. The method of claim 14 where positioning through the body lumen to the location near the heart but within the body lumen comprises positioning a working end of an endoscope to the location.

19. The method of claim 14 where the creating steps further involve sealing the opening to keep fluid from flowing into or out of the opening.

20. The method of claim 14 where the second navigation step involves the creation of artificial pathways from the lumen wall to the surface using tissue dissection or removal.

21. The method of claim 14 where the second navigation step involves the creation of a working space to allow deployment and manipulation of devices and visualize.

22. The method of claim 14 where the closure steps are accomplished through methods selected from known mechanical, electrical, microwave, laser, thermal or chemical techniques.

23. The method of claim 14 where the closure steps involve the application of one or more drugs to aid healing.

24. The method of claim 14 where the devices are manipulated remotely or robotically.

25. The method of claim 14 where more than one opening in a wall of a body lumen is created to execute the procedure.

26. The method of claim 14 where one or more openings are created in more than one body lumen to execute the procedure.
